(19) United States
(12) Plant Patent Application Publication
(10) Pub. No.: US 2007/0186319 P1
(43) Pub. Date: Aug. 9, 2007

(54) Hydrangea plant named 'Hopcorn'

(76) Inventors: Jacobus Gerardus Hofstede, Huissen (NL); Wilhelmus Jacobus Gerardus Hofstede, Huissen (NL)

Correspondence Address: JONDLE & ASSOCIATES P.C., 858 HAPPY CANYON ROAD SUITE 230, CASTLE ROCK, CO, 80108, US

(21) Appl. No.: 11/349,678

(22) Filed: Feb. 8, 2006

(51) Int. Cl. A01H 5/00

(52) U.S. Cl. PLT/250

(57) ABSTRACT

A hydrangea cultivar particularly distinguished by its bright pink inflorescences with cupped sepals, dark green leaves, and vigorous growth habit is disclosed.

### GENUS AND SPECIES

Hydrangea macrophylla

### VARIETY DENOMINATION

‘Hopcorn’

### BACKGROUND OF THE NEW PLANT

The present invention comprises a new and distinct cultivar of hydrangea, botanically known as Hydrangea macrophylla, and hereinafter referred to by the cultivar name ‘Hopcorn’. The new cultivar originated from a naturally occurring mutation of the hydrangea cultivar ‘Mathilde Gutges’ (unpatented) found in 1998 in Huissen, The Netherlands. The flowers of the mutant plant had smaller sepals and each sepal was cupped, unlike the normal flat sepals of the mutation parent plant.

Like the mutation parent, ‘Hopcorn’ has pink flowers which can turn blue when an aluminum treatment has been applied in combination with growing the plant in acid soil. Hydrangea cultivar ‘Hopcorn’ has been asexually reproduced repeatedly by vegetative cuttings in Huissen, The Netherlands over a five-year period. ‘Hopcorn’ has been found to retain its distinctive characteristics through successive asexual propagations.

### DESCRIPTION OF PHOTOGRAPHS

This new hydrangea plant is illustrated by the accompanying photographs which show blooms, buds, and foliage of the plant; the colors shown are as true as can be reasonably obtained by conventional photographic procedures. The photographs are of a three-year old plant grown in a greenhouse.

FIG. 1 shows overall plant habit, including blooms and foliage.

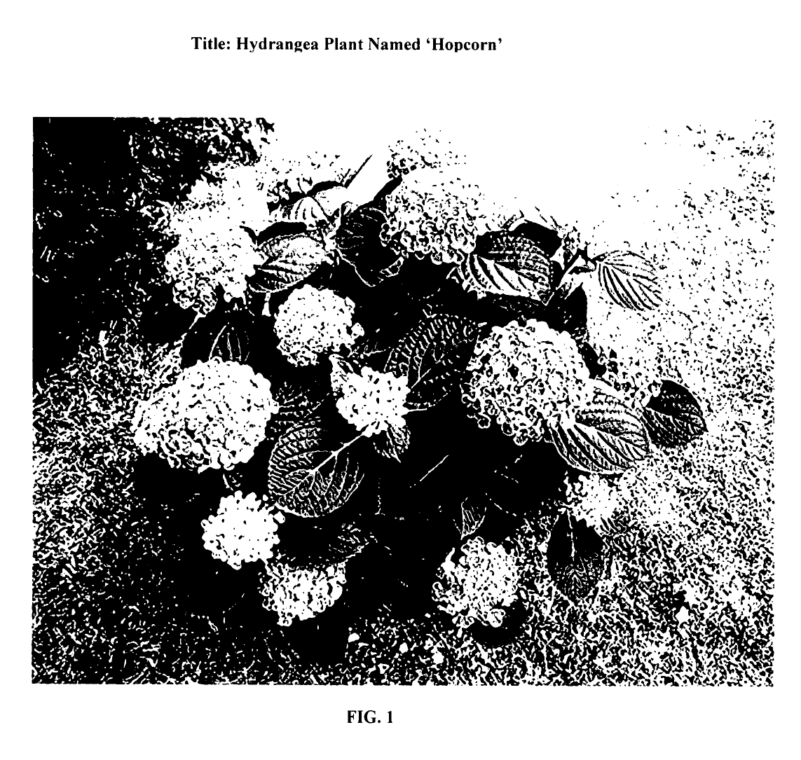

FIG. 2 shows a close-up of a mature inflorescence.

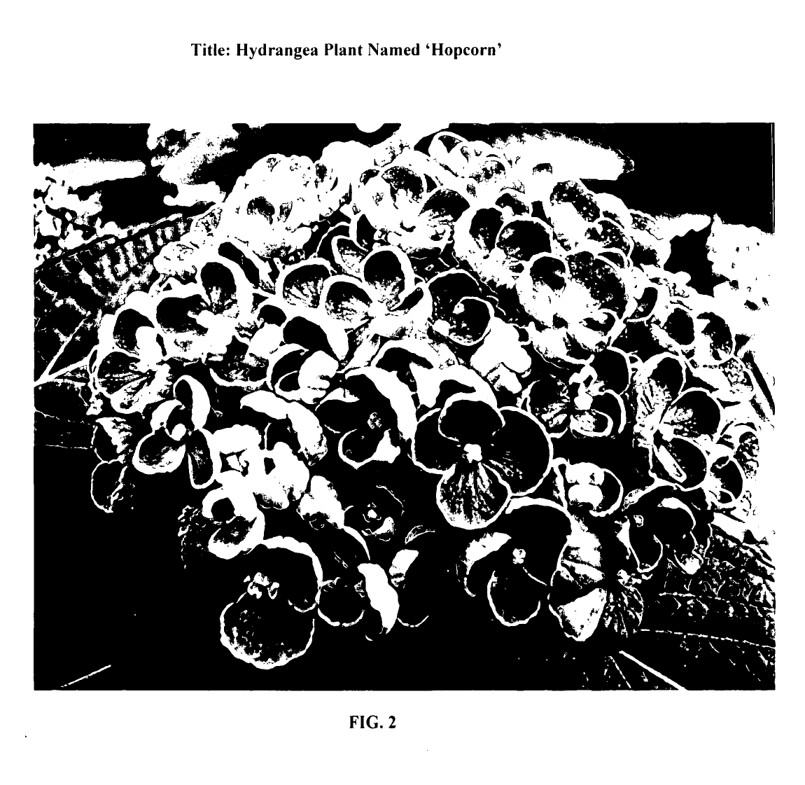

### DESCRIPTION OF THE NEW CULTIVAR

The following detailed descriptions set forth the distinctive characteristics of ‘Hopcorn’. The data which define these characteristics were collected from asexual reproductions carried out in Huissen, The Netherlands. The plant history was taken on eighteen-month-old plants grown in three-liter pots, two cuttings per pot, in a greenhouse. Color references are primarily to The R.H.S. Colour Chart of The Royal Horticultural Society of London (R.H.S.) (2001).

### DETAILED BOTANICAL DESCRIPTION

Classification:
   Botanical.—Hydrangea macrophylla.
   Common Name.—Hydrangea.
Parentage: Naturally occurring mutation of hydrangea cultivar ‘Mathilde Gutges’.
Growth:
   Habit.—Medium to tall growth habit; growth retarders are used to keep plants shorter; average sturdiness; 10 to 12 branches with flowers.
   Height.—40 cm to 50 cm.
   Width.—35 cm to 45 cm.
Stems:
   General.—Stems become woody as they age.
   Young stems.—Color: R.H.S. 139C Diameter: 0.4 cm to 0.6 cm Internode length: Strongly dependent on usage of growth retarders; on a plant of 40 cm height the internode length averages 5.5 cm Shape: Round Texture: Glabrous Pubescence: None Odor: Not different from other H. macrophylla Pith: Type: Solid Diameter: 0.35 cm (as measured one-half way from apex to start of one year's growth) Color: R.H.S. 155C and R.H.S. 157D Lenticels: Number: 15 to 20 per centimeter of stem Shape: Oblong Size: Length: 0.05 cm to 0.25 cm Width: 0.03 cm to 0.05 cm Color: Near R.H.S. 79B and R.H.S. 86A Orientation: Vertical in the direction of the length of the stem.
   Mature stems.—Color: Thin stems: R.H.S. 177A, R.H.S. 197A, and R.H.S. 200D Thick stems: Near R.H.S. 201C and R.H.S. 198A Diameter: Thin stems: 0.4 cm to 0.5 cm Thick stems: 0.55 cm to 0.65 cm Exfoliation: Thin stems: 0.1 cm to 0.2 cm cracks in the bark Thick stems: 0.3 cm to 0.4 cm cracks in the bark.
Leaves:
   Arrangement.—Opposite.
   Color.—Young leaves: Upper surface: R.H.S. 137A Lower surface: R.H.S. 137C and R.H.S. 138A Mature leaves: Upper surface: R.H.S. 136A Lower surface: R.H.S. 139B and R.H.S. 138A.
   Mature leaf size.—Length: 13 cm to 15 cm Width: 9.5 cm to 10.5 cm.
   Apex.—Acute.
   Base.—Cuneate to obtuse.
   Shape.—Elliptic to obovate.
   Margin.—Serrulate.
   Texture.—Upper surface: Glabrous Lower surface: Very few small hairs on main vein and large side veins.
   Venation.—10 to 12 large prominent side veins coming from the main vein.
   Vein color.—Near R.H.S. 138B and R.H.S. 139C.
   Petioles.—Length: 2.8 cm to 3.8 cm Diameter: 0.4 cm to 0.5 cm Color: Near R.H.S. 138B and R.H.S. 139C Texture: Glabrous Shape: U-shaped with groove above.
Inflorescence:
   Type.—Compound corymb, semi-globose.
   Diameter.—11 cm to 15 cm, rounded.
   Depth (height).—6 cm to 8 cm.
   Number of individual flowers per inflorescence.—50 to 60 sterile flowers in an 11 cm-sized inflorescence; 110 to 120 sterile flowers in a 16 cm-sized inflorescence.
   Blooming habit.—Blooms from June through September.
   Color.—At emergence (end of June): Near R.H.S. 63B and R.H.S. 64C At full bloom (mid-July): Near R.H.S. 59D, R.H.S. 60C and R.H.S. 61B At fading (September): R.H.S. 186A.
   Peduncle.—Color: R.H.S. 143A.
   Pedicel.—Color: Near R.H.S. 70C.
Petals:
   Petal color (at full bloom).—Upper surface: Near R.H.S. 70D and R.H.S. 75B Lower surface: Near R.H.S. 64D and R.H.S. 68A.
Sepals:
   Number of sepals.—4 per sterile flower.
   Size, small sterile flowers.—Length: 0.9 cm to 1.1 cm Width: 0.9 cm to 1.1 cm.
   Size, large sterile flowers.—Length: 1.4 cm to 1.6 cm Width: 1.4 cm to 1.6 cm.
   Shape.—Cupped; plants forced into flower very early in the season can have sepals that are more cupped than plants that flower later in the season.
   Apex.—Round.
   Base.—Obtuse.
   Margin.—Round and smooth when grown in a heated greenhouse; obtuse on the upper half of the sepal, lower half no incisions, when grown in a cold greenhouse or outside.
   Texture.—Glabrous.
   Color (at full bloom).—Upper surface: Near R.H.S. 59D, R.H.S. 60C and R.H.S. 61B Lower surface: Near R.H.S. 64D and R.H.S. 68A.
Reproductive organs:
   Anther.—Size: Length: 0.1 cm Width: 0.1 cm Depth: 0.05 cm Color: Near R.H.S. 70D and R.H.S. 75B.
   Filament.—Color: Near R.H.S. 65B, R.H.S. 63C, and R.H.S. 62B Size: Length: 0.3 cm Width: Too thin to measure Pollen color: Near R.H.S. 155C and R.H.S. 155D.
   Pistil.—Stigma color: R.H.S. 62C Style color: R.H.S. 63A and R.H.S. 64B.
   Fruit set.—None observed.
   Seed.—None observed.
Disease and insect resistance: ‘Hopcorn’ is less sensitive than average H. macrophylla for Botrytis during winter storage.

### COMPARISON WITH KNOWN CULTIVARS

Cultivar ‘Hopcorn’ differs from the mutation parent ‘Mathilde Gutges’ (unpatented) by having smaller sepals than ‘Mathilde Gutges’ and by having each sepal cupped while ‘Mathilde Gutges’ has the normal flat sepals.

Cultivar ‘Hopcorn’ differs from the commercial variety, ‘Uzu’ (unpatented) by having darker leaves and darker pink flowers. Additionally, ‘Hopcorn’ also grows more compactly than ‘Uzu’.

I claim:

1. A new and distinct cultivar of hydrangea plant as shown and described herein.
